(19) United States
(12) Patent Application Publication
(10) Pub. No.: US 2007/0129599 A1
(43) Pub. Date: Jun. 7, 2007

(54) Massage apparatus having a connection system

(75) Inventor: Simon Siu Man Nan, Richmond Hill (CA)

Correspondence Address: GREENSFELDER HEMKER & GALE PC, SUITE 2000, 10 SOUTH BROADWAY, ST LOUIS, MO, 63102, US

(73) Assignee: NANMA MANUFACTURING CO. LTD., Chai Wan (HK)

(21) Appl. No.: 11/296,525

(22) Filed: Dec. 7, 2005

(51) Int. Cl. A61F 5/00; A61H 1/00

(52) U.S. Cl. 600/38; 601/70

(57) ABSTRACT

A massage apparatus having a connection system for exchanging a prosthetic member is disclosed. The prosthetic member is adapted to receive an outer sleeve therein for providing a sleeve assembly that collectively engages an inner casing defining a chamber having a vibratory component disposed therein. The vibratory component provides a vibrating function and a control cap in operative association with the vibratory component in order to control the operation of the vibratory component. The outer sleeve may define an arrangement of slots and protrusions defined around the outer circumference of the outer sleeve that are adapted to engage respective segments defined around the periphery of the inner casing such that the outer sleeve becomes engaged with the inner casing when the user rotates the outer sleeve relative to the inner casing.

### FIELD

The present document relates to a massage apparatus, and more particularly to a massage apparatus having a connection system.

### SUMMARY

In an embodiment, the massage apparatus comprises a prosthetic member defining an outer chamber, a hollow outer sleeve adapted to be engaged inside the outer chamber of the prosthetic member, and an inner casing adapted to be engaged inside the hollow outer sleeve, wherein the hollow outer sleeve and the inner casing include a connection system for engaging the hollow outer sleeve to the inner casing the hollow outer sleeve being adapted to engage the inner casing such that the prosthetic member may be engaged or disengaged with the inner casing.

In another embodiment, the massage apparatus comprises a prosthetic member defining an outer chamber, a hollow outer sleeve adapted to be engaged inside the outer chamber of the prosthetic member, and an inner casing engaged inside the hollow outer sleeve, wherein the outer sleeve defines a plurality of slots and the inner casing defines a plurality of segments adapted to engage the plurality of slots when engaging the outer sleeve to the inner casing.

In one embodiment, the massage apparatus comprises a sleeve assembly including a prosthetic member adapted to be engaged inside a hollow outer sleeve, and an inner casing adapted to be engaged inside the sleeve assembly, wherein the sleeve assembly being adapted to be engaged to the inner casing, wherein the hollow outer sleeve defines a plurality of slots and the inner casing defines a plurality of segments adapted to engage the plurality of slots when engaging the sleeve assembly to the inner casing.

In one embodiment, a method of exchanging external features of a massage apparatus comprising:

a) providing a massage apparatus comprising a sleeve assembly including a hollow outer sleeve adapted to be engaged inside a prosthetic member, and an inner casing adapted to be engaged inside the sleeve assembly, wherein the hollow outer sleeve defines a plurality of slots and the inner casing defines a plurality of segments adapted to engage the plurality of slots when engaging the sleeve assembly to the inner casing;

b) disengaging the plurality of slots from the plurality of segments such that the sleeve assembly is disengaged from the inner casing; and

c) engaging a plurality of slots of another hollow outer sleeve to the plurality of segments of the inner casing such that another sleeve assembly is engaged to the inner casing.

Implementations of the above embodiments may include one or more of the following features:

The massage apparatus further comprises a vibratory component disposed inside the inner casing and in operative association with a power source, the vibratory component providing a vibration function when in operative association with the power source.

The connection system includes a plurality of slots defined by the hollow outer sleeve and a plurality of segments defined by the inner casing, the plurality of segments being adapted to engage the plurality of slots when engaging the hollow outer sleeve to the inner casing.

The inner casing defines one or more ribs adapted to be in biased contact with the hollow outer sleeve when the inner casing is engaged to the hollow outer sleeve.

The plurality of segments are spaced equidistant from one another.

The hollow outer sleeve defines a shoulder, the shoulder defining a plurality of slots.

The inner casing includes a flange, the flange defining a plurality of segments.

The flange is adapted to engage the shoulder of the hollow outer sleeve.

The plurality of slots are adapted to engage the plurality of segments when engaging the shoulder to the flange.

The step of disengaging the plurality of slots from the plurality of segments comprises rotating the inner casing relative to the hollow outer sleeve and then sliding the hollow outer sleeve away from the inner casing.

The step of engaging a plurality of slots of another hollow outer sleeve to the plurality of segments of the inner casing comprises sliding the hollow outer sleeve towards the inner casing and then rotating the inner casing relative to the hollow outer sleeve.

The method further comprising the step of disengaging the prosthetic member from the outer sleeve and engaging another prosthetic member to the outer sleeve.

Additional features will be set forth in the description which follows, and will become apparent to those skilled in the art upon examination of the following more detailed description and drawings in which like elements of the invention are similarly numbered throughout.

### BRIEF DESCRIPTION OF THE DRAWINGS

FIG. 1 is a perspective view of the massage apparatus;

FIG. 2 is a cross-sectional view of the massage apparatus taken along line 2-2 of FIG. 1;

FIG. 3 is a cross-sectional view of the massage apparatus without the prosthetic member;

FIG. 4 is a partial cross-sectional view of the massage apparatus;

FIG. 5 is an end view of the massage apparatus taken along line 5-5 of FIG. 4;

FIG. 6 is a cross-sectional view of the massage apparatus taken along line 6-6 of FIG. 4; and

FIG. 7 is a cross-sectional view of the massage apparatus taken along line 7-7 of FIG. 3.

Corresponding reference characters indicate corresponding elements among the view of the drawings.

### DETAILED DESCRIPTION

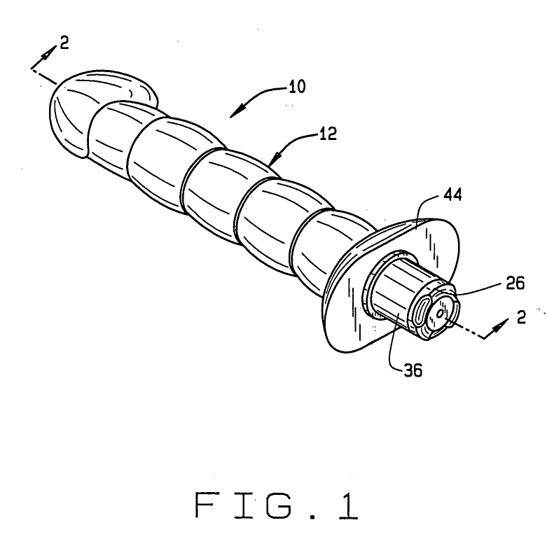

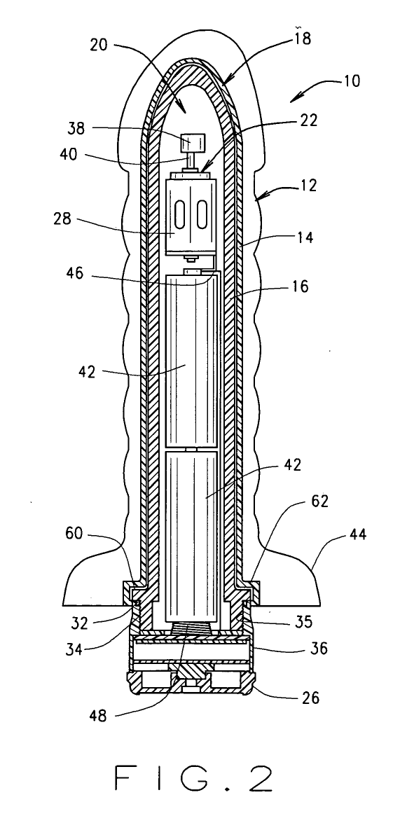

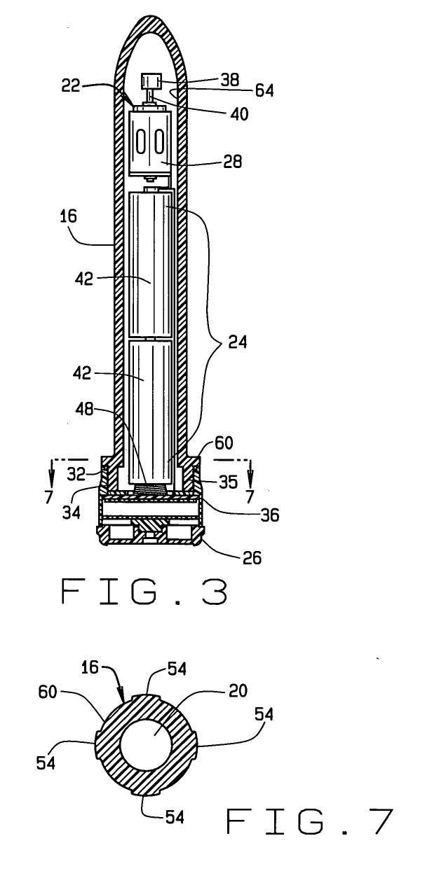

Referring to the drawings, an embodiment of a massage apparatus is illustrated and generally indicated as 10 in FIGS. 1-7. Referring to FIGS. 1 and 2, the massage apparatus 10 may include an inner casing 16 having a chamber. The chamber 20 of inner casing 16 may be adapted to receive a vibratory component 22 in operative association with a power source 24 (FIG. 3) through an electrical circuit 46 for providing a vibrating function that vibrates the massage apparatus 10. Referring to FIG. 3, power source 24 may be one or more batteries 42 with at least one of the batteries 42 in operative engagement with a spring 48 which is operatively associated with electrical circuit 46. However, other suitable sources of power for massage apparatus 10 are contemplated, such as a single battery or an electrical outlet connection. The inner casing 16 may further include a flange 60 adapted to be engaged within a hollow outer sleeve 14 having a shoulder 62 that collectively form a connection system for engaging and disengaging the hollow outer sleeve 14 from the inner casing 16 as shall be discussed below.

In one aspect, a prosthetic member 12 defining an outer chamber 18 may be adapted to engage the hollow outer sleeve 14 inside outer chamber 18. In one embodiment the prosthetic member 12 may be adapted to be interchangeable by disengaging the outer sleeve 14 engaged to one prosthetic member 12 from inner casing 16 and engaging another outer sleeve 14 engaged to another prosthetic member 12. Prosthetic member 12 to inner casing 16 as shall be discussed below.

In an embodiment, the prosthetic member 12 may have a generally phallic shape, although other shapes are contemplated, such as segmented, smooth, rounded, bumpy, tubular and/or angular configurations. The prosthetic member 12 may be an exchangeable external feature made of a resilient material, such as plastic, rubber, or polymer material, that forms the outermost feature of the massage apparatus 10. In one embodiment, the prosthetic member 12 may define a flange 44, although other embodiments may lack the flange 44.

Referring specifically to FIG. 2, the inner casing 16 may be adapted to engage a battery cover 36 for encasing batteries 42. The battery cover 36 may define internal threads 35 adapted to engage external threads 34 defined by inner casing 16 such that the battery cover 36 may be engaged to the inner casing 16. A sealing element 32 made of a resilient material may be interposed between the battery cover 36 and the inner casing 16 for providing a water-tight seal between the two components.

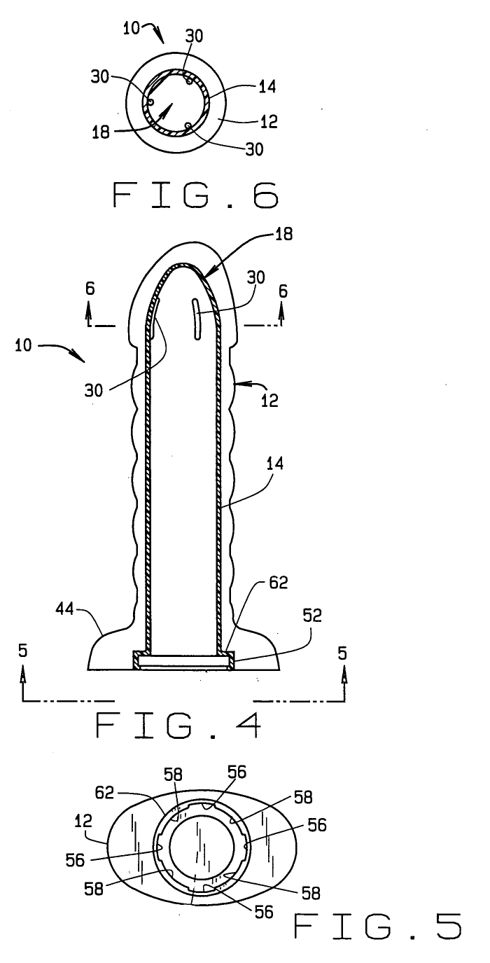

Referring to FIG. 4, the hollow outer sleeve 14 includes a body 52 that may define a plurality of longitudinal ribs 30 that protrude inwardly along the internal circumference of the outer sleeve 14. Upon engaging the inner casing 16 to the hollow outer sleeve 14, the longitudinal ribs 30 establish a biased contact between the inner casing 16 and the hollow outer sleeve 14. In one embodiment, three longitudinal ribs 30 are spaced equidistant along the internal circumference of the hollow outer sleeve 14, although one or more longitudinal ribs 30 spaced along the hollow outer sleeve 14 are contemplated.

Referring back to FIG. 3, the vibratory component 22 provides a vibrating function that vibrates the massage apparatus 10 when made operational by the user. The vibratory component 22 may include a motor 28 in operational engagement with a rod 40 that is rotated and spins an eccentric mass 38 mounted on rod 40 which causes inner casing 16 to vibrate. A control cap 26 may be engaged to the battery cover 36 and operatively associated with the vibratory component 22 for controlling the vibrating function of the vibratory component 22.

Referring to FIG. 5, the shoulder 62 of outer sleeve 14 may define a plurality of alternating slots 56 and inward protrusions 58. In one embodiment, the alternating slots 56 and protrusions 58 may be spaced equidistant in alternating fashion along the inner circumference of shoulder 62 such that slots 56 provide one portion of the connection system adapted to engage the hollow outer sleeve 14 to inner casing 16 as shall be discussed in greater detail below.

As shown in FIG. 7, the flange 60 of inner casing 16 may include a plurality of segments 54 defined around the periphery of flange 60. In one embodiment, segments 54 may be adapted to engage slots 56 in order to engage hollow outer sleeve 14 to the inner casing 16. In one embodiment, the segments 54 may be spaced around the periphery of flange 60 such that each segment 54 may engage a respective slot 56 when the inner casing 16 is engaged to the hollow outer sleeve 14. In one aspect, when the prosthetic member 12 is engaged to the hollow outer sleeve 14 a sleeve assembly is formed that provides the capability to exchange various shaped prosthetic members 12 with the massage apparatus 10 by simply disengaging the hollow outer sleeve 14 from the inner casing 16 and engaging another hollow outer sleeve 14 to inner casing 16. However, in another embodiment prosthetic member 12 may be directly disengaged from the outer sleeve 14 and another prosthetic member 12 engaged to outer sleeve 14.

The method of exchanging the sleeve assembly will now be discussed. The sleeve assembly may be engaged to the inner casing 16 by sliding the inner casing 16 inside the hollow outer sleeve 14 of the sleeve assembly such that the slots 56 of the hollow outer sleeve 14 are properly aligned with respective segments 54 of inner casing 16. Once the inner casing 16 is engaged inside the outer sleeve 14, the outer sleeve 14 is then rotated relative to the inner casing 16 which engages the sleeve assembly to inner casing 16 as the slots 56 of the outer sleeve 14 engage segments 54 of the inner casing 16. This engagement between the segments 54 and slots 56 will cause the longitudinal ribs 30 to bias and closely contact the outer surface of inner casing 16 along portion 64 such that the vibrating function generated by the vibratory component 22 inside the inner casing 16 can be transferred to the sleeve assembly.

Conversely, rotating the outer sleeve 14 in an opposite direction relative to the inner casing 16 causes the slots 56 of outer sleeve 14 to disengage the segments 54 of inner casing 16 such that the outer sleeve 14 may be slid away from the inner casing 16, thereby permitting the sleeve assembly to be removed from inner casing 16. The user may then engage another prosthetic member 12 to the inner casing 16 by disengaging the previous prosthetic member 12 from the hollow outer sleeve 14 and engaging another prosthetic member 12 thereto and then engaging the newly formed sleeve assembly to the inner casing 16. Alternatively, another sleeve assembly having a different prosthetic member 12 may be engaged to the inner casing 16 rather than engaging another prosthetic member 12 to the existing outer sleeve 14. Various prosthetic members 12 may be interchanged with the inner casing 16 using the methods discussed above.

It should be understood from the foregoing that, while particular embodiments have been illustrated and described, various modifications can be made thereto without departing from the spirit and scope of the present invention as will be apparent to those skilled in the art. Such changes and modifications are within the scope and teaching of this invention as defined by the claims appended hereto is intended to be limited only by the appended claims.

What is claimed:

1. A massage apparatus comprising:
a prosthetic member defining an outer chamber,
a hollow outer sleeve adapted to be engaged inside said outer chamber of said prosthetic member, and
an inner casing adapted to be engaged inside said hollow outer sleeve, wherein said hollow outer sleeve and said inner casing include a connection system for engaging the hollow outer sleeve to the inner casing, said connection system being adapted to engage said inner casing with said hollow outer sleeve such that said prosthetic member may be engaged or disengaged with said inner casing.

2. The massage apparatus according to claim 1, further comprising a vibratory component disposed inside said inner casing and in operative association with a power source, said vibratory component providing a vibration function when in operative association with said power source.

3. The massage apparatus according to claim 1, wherein said connection system includes a plurality of slots defined by said hollow outer sleeve and a plurality of segments defined by said inner casing, said plurality of segments being adapted to engage said plurality of slots when engaging said hollow outer sleeve to said inner casing.

4. The massage apparatus according to claim 1, wherein said inner casing defines one or more ribs adapted to be in biased contact with said hollow outer sleeve when said inner casing is engaged to said hollow outer sleeve.

5. The massage apparatus according to claim 2, wherein said plurality of segments are spaced equidistant from one another.

6. The massage apparatus according to claim 1, wherein said hollow outer sleeve defines a shoulder, said shoulder defining a plurality of slots.

7. The massage apparatus according to claim 6, wherein said inner casing includes a flange, said flange defining a plurality of segments.

8. The massage apparatus according to claim 7, wherein said flange is adapted to engage said shoulder of said hollow outer sleeve.

9. The massage apparatus according to claim 8 wherein said plurality of slots are adapted to engage said plurality of segments when engaging said shoulder to said flange.

10. A massage apparatus comprising:
a prosthetic member defining an outer chamber,
a hollow outer sleeve adapted to be engaged inside said outer chamber of said prosthetic member, and
an inner casing adapted to be engaged inside said hollow outer sleeve, wherein said outer sleeve defines a plurality of slots and said inner casing defines a plurality of segments adapted to engage said plurality of slots when said hollow outer sleeve is engaged to said inner casing.

11. A massage apparatus comprising:
a sleeve assembly including a prosthetic member adapted to be engaged inside a hollow outer sleeve, and
an inner casing adapted to be engaged inside said sleeve assembly, wherein said sleeve assembly being adapted to be engaged to said inner casing, wherein said hollow outer sleeve defines a plurality of slots, and said inner casing defines a plurality of segments adapted to engage said plurality of slots when engaging said sleeve assembly to said inner casing.

12. A method of exchanging external features of a massage apparatus comprising:
a) providing a massage apparatus comprising a sleeve assembly including a hollow outer sleeve adapted to be engaged inside a prosthetic member, and an inner casing adapted to be engaged inside said sleeve assembly, wherein said hollow outer sleeve defines a plurality of slots and said inner casing defines a plurality of segments adapted to engage said plurality of slots when engaging said sleeve assembly to said inner casing;
b) disengaging the plurality of slots from the plurality of segments such that the sleeve assembly is disengaged from the inner casing; and
c) engaging the plurality of slots of another hollow outer sleeve to the plurality of segments of said inner casing such that another sleeve assembly is engaged to the inner casing.

13. The method according to claim 12, wherein the step of disengaging the plurality of slots from the plurality of segments comprises rotating the inner casing relative to the hollow outer sleeve and then sliding the hollow outer sleeve away from the inner casing.

14. The method according to claim 12, wherein the step of engaging a plurality of slots of another hollow outer sleeve to the plurality of segments of the inner casing comprises sliding the hollow outer sleeve towards the inner casing and then rotating the inner casing relative to the hollow outer sleeve.

15. The method according to claim 12, further comprising the step of disengaging the prosthetic member from the outer sleeve and engaging another prosthetic member to the outer sleeve.
